(19) United States
(12) Patent Application Publication
(10) Pub. No.: US 2007/0128576 A1
(43) Pub. Date: Jun. 7, 2007

(54) Output attachments coded for use with electromagnetic-energy procedural device

(76) Inventor: Dmitri Boutoussov, Dana Point, CA (US)

Correspondence Address: STOUT, UXA, BUYAN & MULLINS LLP, 4 VENTURE, SUITE 300, IRVINE, CA, 92618, US

(21) Appl. No.: 11/231,306

(22) Filed: Sep. 19, 2005

Related U.S. Application Data
(60) Provisional application No. 60/610,757, filed on Sep. 18, 2004.

(51) Int. Cl. A61C 1/00; A61C 3/00; A61B 18/18; A61C 5/00

(52) U.S. Cl. 433/29; 433/215; 606/10

(57) ABSTRACT

Output attachments to an electromagnetic-energy outputting device are coded using colors, topography, or combinations of color and topography. The output attachments, which are removable and interchangeable, may be interchanged for purposes of performing particular surgical or other cutting procedures. Instructions provided to a user refer to colors or other coded indicators in order to provide rapid and reliable switching of output attachments.

### CROSS-REFERENCE TO RELATED APPLICATIONS

This application claims the benefit of U.S. Provisional Application No. 60/610,757, filed Sep. 18, 2004 and entitled OUTPUT ATTACHMENTS CODED FOR USE WITH ELECTROMAGNETIC-ENERGY PROCEDURAL DEVICE, the entire contents of which are hereby incorporated by reference.

### BACKGROUND OF THE INVENTION

1. Field of the Invention

The present invention relates generally to electromagnetic-energy procedural devices and, more particularly, to electromagnetic energy devices used in medical applications.

2. Description of Related Art

Laser devices used in dental and medical applications frequently employ handpieces and attachments that are specialized according to particular medical or dental procedures. For example, a laser can be constructed to output optical energy through a first type of optical waveguide to facilitate implementation of a first procedure, such as dental carries removal, and to output optical energy through a second type of optical waveguide to facilitate implementation of a second procedure, such as tooth whitening. In other cases, a laser base unit including a laser power source capable of providing laser power to a delivery system, may couple to a variety of handpieces and waveguides that deliver laser energy to a treatment site. Ancillary functionality related to power settings, a need for illumination, a need to spray water or air, and other requirements may vary from one handpiece to another depending upon the specifics of a particular treatment plan.

Unintentional or even accidental use of a handpiece or waveguide not properly matched to an intended medical or dental procedure could lead to undesirable consequences including patient discomfort, irreparable damage to treated tissue, lost time, and the like.

A need thus exists in the prior art for methods and apparatus that may provide enhanced assurances regarding proper matching between laser delivery devices and intended treatment techniques. A further need exists for procedures and devices that may facilitate the matching of handpieces and optical waveguides to particular medical or dental applications.

### SUMMARY OF THE INVENTION

The present invention addresses these needs, in accordance with the present invention, using methods and apparatuses that can provide information to a user pertaining to one or more of properties, compatibilities, and procedural suitabilities of various output attachments. The output attachments may possess a common feature of being connectable to an electromagnetic-energy outputting device, such as a laser. These properties, compatibilities, and procedural suitabilities may be communicated to the user by indicia or, in accordance with a feature of the present invention, codes, disposed on one or more of the output attachment, an output end of a handpiece of the electromagnetic-energy output device, and a display of an electromagnetic-energy outputting base unit.

An embodiment of the present invention can include an electromagnetic-energy outputting device comprising an electromagnetic-energy outputting base unit that supplies electromagnetic-energy and a handpiece that receives electromagnetic-energy from the electromagnetic-energy outputting base unit and accepts one or more of a plurality of output attachments. The embodiment further can comprise a plurality of output attachments that can be coupled to the handpiece, wherein a coupled output attachment can receive electromagnetic-energy from the handpiece and deliver electromagnetic-energy to a target site. In one embodiment of the electromagnetic-energy outputting device, each one of the plurality of output attachments is distinguishable from another one of the plurality of output attachments according to one or more of a physically-discernable property and a functional property. Each one of the plurality of output attachments is coded according to one or more of the physically-discernable property and the functional property.

Another embodiment of the present invention comprises a code describing interchangeable attachments to an electromagnetic-energy outputting device, wherein the code comprises a list of attachments and a list of colors placed into one-to-one correspondence with the attachments. The attachments are distinguishable according to one or more of a physically discernable property and a functional property. The one-to-one correspondence is selected according to one or more of the physically-discernable property and the functional property.

An implementation of the present invention can provide a method of coding interchangeable output attachments that attach to an electromagnetic-energy outputting device. One example of the method can comprise creating a list of output attachments, wherein one of the attachments in the list is distinguishable from another output attachment in the list according to a physically-discernable property. The example further can comprise creating a list of colors and placing the colors into one-to-one correspondence with the output attachments.

Another implementation of the present invention provides a method of operating an electromagnetic-energy outputting device, the method comprising receiving at least one coded instruction and connecting an output attachment to the electromagnetic-energy outputting device according to the coded instruction.

While the apparatus and method have or will be described for the sake of grammatical fluidity with functional explanations, it is to be expressly understood that the claims, unless expressly formulated under 35 U.S.C. 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. 112 are to be accorded full statutory equivalents under 35 U.S.C. 112.

Any feature or combination of features described herein are included within the scope of the present invention provided that the features included in any such combination are not mutually inconsistent as will be apparent from the context, this specification, and the knowledge of one skilled in the art. For purposes of summarizing the present invention, certain aspects, advantages and novel features of the present invention are described herein. Of course, it is to be understood that not necessarily all such aspects, advantages or features will be embodied in any particular embodiment of the present invention. Additional advantages and aspects of the present invention are apparent in the following detailed description and claims that follow.

### BRIEF DESCRIPTION OF THE FIGURES

FIG. 1 is a pictorial diagram of a delivery system capable of transferring electromagnetic energy to a treatment site;

FIG. 2 is a cross-section of a distal portion of an electromagnetic-energy outputting handpiece showing a sleeve and a fiber tip;

FIG. 3 is an on-axis view of a combination formed by a sleeve and a fiber tip as depicted in FIG. 2;

FIG. 4 is a pictorial diagram of an output attachment according to the present invention;

FIG. 5 is a pictorial diagram of a tip structure;

FIG. 6 is a flow diagram describing an exemplary operation that may be performed using an electromagnetic-energy outputting device;

FIG. 7 is a flow diagram depicting the operation of FIG. 6 employing a method of the present invention that may address or overcome limitations of the prior art;

FIG. 8 is a flow diagram describing an implementation of a method of creating a code according to the present invention;

FIG. 9 is a pictorial diagram of an embodiment of a conduit shown in FIG. 1;

FIG. 10 is a partial cut-away diagram of a handpiece tip;

FIG. 10a is a pictorial diagram of detail of the handpiece tip of FIG. 10 illustrating a mixing chamber for spray air and water;

FIG. 11 is a sectional view of a proximal member of FIG. 9 taken along line 11-11′ of FIG. 9;

FIG. 12 is a cross-sectional view of a handpiece tip taken along line 12-12′ of FIG. 10;

FIG. 13 is a cross-sectional diagram of another embodiment of the handpiece tip taken along the line 12-12′ of FIG. 10; and

FIG. 14 is a cross-sectional diagram of another embodiment of the handpiece tip taken along line 14-14′ of FIG. 10.

### DETAILED DESCRIPTION OF THE INVENTION

Reference will now be made in detail to the presently preferred embodiments of the invention, examples of which are illustrated in the accompanying drawings. Wherever possible, the same or similar reference numbers are used in the drawings and the description to refer to the same or like parts. It should be noted that the drawings are in simplified form and are not to precise scale. In reference to the disclosure herein, for purposes of convenience and clarity only, directional terms, such as, top, bottom, left, right, up, down, over, above, below, beneath, rear, and front, are used with respect to the accompanying drawings. Such directional terms should not be construed to limit the scope of the invention in any manner.

Although the disclosure herein refers to certain illustrated embodiments, it is to be understood that these embodiments are presented by way of example and not by way of limitation. The intent of the following detailed description, although discussing exemplary embodiments, is to be construed to cover all modifications, alternatives, and equivalents of the embodiments as may fall within the spirit and scope of the invention as defined by the appended claims. It is to be understood and appreciated that the process steps and structures described herein do not cover a complete process flow for operation of electromagnetic-energy outputting devices. The present invention may be practiced in conjunction with various techniques that are conventionally used in the art, and only so much of the commonly practiced process steps are included herein as are necessary to provide an understanding of the present invention. The present invention has applicability in the field of electromagnetic energy devices in general. For illustrative purposes, however, the following description pertains to a medical electromagnetic-energy outputting device and a method of operating the medical electromagnetic-energy outputting device to perform surgical functions.

In accordance with an aspect of the present invention, a plurality of output attachments is constructed to be coupled to an electromagnetic-energy outputting handpiece. The output attachments may differ one from another in one or more of a physically-discernable property and a functional property. According to an exemplary embodiment, the output attachments are color coded (e.g., coded according to a color pattern) according to a property (e.g., a physically-discernable or functional property) of the output attachment. According to another embodiment, the output attachments are topography coded (e.g., topography-pattern coded) to designate varying properties (e.g., varying physically-discernable or functional properties) among the output attachments. Still another embodiment codes the output attachments according to a color pattern and a topography pattern.

A physically-discernable property of an output attachment can comprise, for example, a diameter of a waveguide of the output attachment. A functional property can comprise, for example, a rating such as a rated or recommended number of pulses received by a waveguide of the output attachment before replacement.

Color codes can comprise colors that do not vary on a given output attachment (e.g., solid patches of color). Color codes may be associated with different output attachments according to different properties of the output attachments. Color codes can be patterned, in which case the codes may comprise colors that vary on a given output attachment (e.g., a non-solid color or a combination of colors). Colors also may be chosen to distinguish among different output attachments having different properties according to the different properties.

Topography codes can comprise tactile and/or visual surface disturbances. In representative embodiments, topography codes comprise various surface disturbances or patterns of surface disturbances that vary among different output attachments. Differences among the topography codes of the different output attachments may correspond to or may designate differences among properties of the output attachments. Topography codes can comprise, for example, non-alphanumeric surface disturbances that provide for one or more of tactile and visual differentiation among the different properties of the output attachments. The non-alphanumeric surface disturbances can comprise, for example, raised shapes (e.g., stripes or circles), sunken shapes, or other features or patterns of features, having different dimensions and/or orientations relative to one another on a given output attachment or relative to one another among output attachments with varying properties. The topography codes can further comprise, for example, non-machine readable surface disturbances that differentiate to a user varying properties of the output attachments. Machine-readable codes can comprise, for example, bar codes and alphanumerics. In one embodiment, surface disturbances or patterns thereof (which may comprise, for example, alphanumeric surface disturbances and which may or may not differ among various output attachments) can be combined with colors or patterns thereof to facilitate differentiation among the varying properties of the output attachments. In one implementation, heights and/or depths defining one or more topography codes can be varied. In a set of topography codes, one or more of the codes may be varied along one or more dimensions while other ones of the set (on a single output attachment or among a set of output attachments) are not varied. In a modified embodiment, the surface disturbances can be combined with differences in color to indicate the different properties of the output attachments.

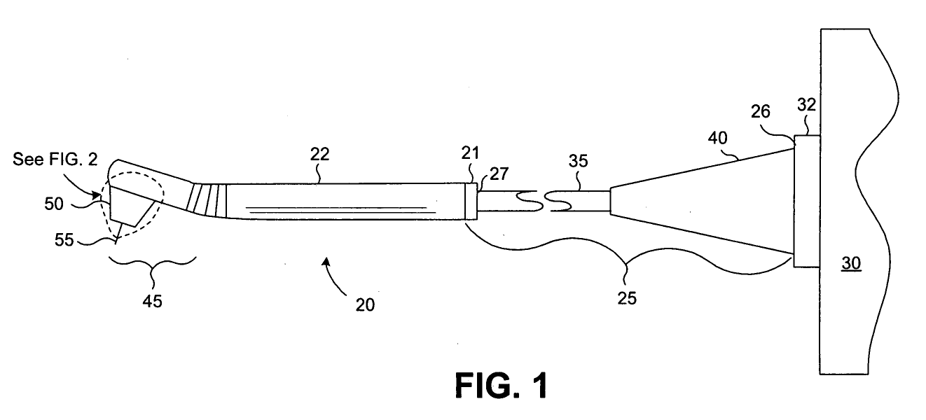

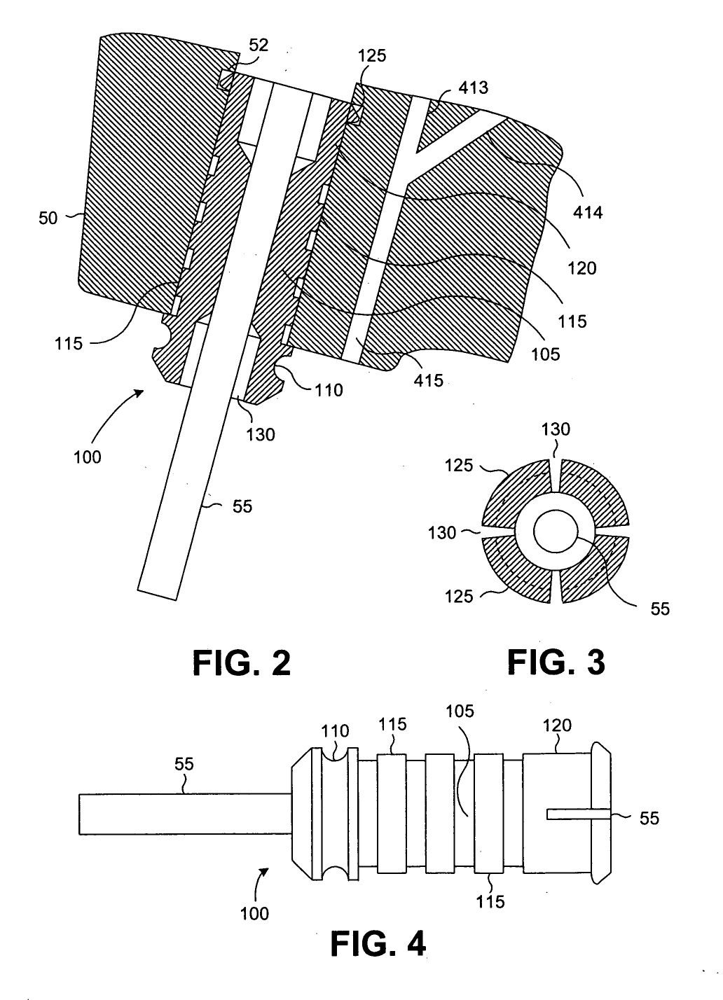

Referring more particularly to the drawings, FIG. 1 is a pictorial diagram of a delivery system of an electromagnetic-energy procedural device that is capable of transferring electromagnetic energy to a treatment site. The illustrated embodiment comprises an electromagnetic-energy outputting handpiece 20 that connects to an electromagnetic-energy outputting base unit 30 using a linking element 25. The electromagnetic-energy outputting handpiece 20 may comprise an elongate portion 22 and a handpiece tip 45. A proximal (i.e., relatively nearer to the electromagnetic-energy outputting base unit 30) portion 21 and a distal (i.e., relatively further from the electromagnetic-energy outputting base unit 30) portion 50 may be disposed at respective proximal and distal regions of the electromagnetic-energy outputting handpiece 20. The distal portion 50 has protruding therefrom a fiber tip 55, which is shown in greater detail in FIG. 2.

The linking element 25 may comprise a first end 26 and a second end 27. First end 26 is shown in FIG. 1 as coupling to a receptacle 32 of the electromagnetic-energy outputting base unit 30. Second end 27 couples to the proximal portion 21 of the electromagnetic-energy outputting handpiece 20 in the illustrated embodiment. The linking element 25 may further include a conduit 35, which may comprise one or more optical fibers, tubing for air, tubing for water, and the like (not shown) and a connector 40 that joins the conduit 35 to the electromagnetic-energy outputting base unit 30.

An implementation of the present invention features output attachments, each comprising a housing coupled to a waveguide, wherein the housing and similar housings may be color or topography coded according to one or more different properties of respective waveguides to which they are attached. For instance, the housings may be color coded to indicate different magnitudes of a particular property (e.g., dimension) of the output-waveguides held by the respective housings.

According to a further implementation, for an exemplary output attachment 100 shown in FIG. 2, the housing may comprise a sleeve 105 supported by the distal portion 50 of the electromagnetic-energy outputting handpiece 20 (FIG. 1), and the waveguide may comprise a fiber tip 55. A typical output attachment may comprise, as an example, both a sleeve 105 and a fiber tip 55 held by the sleeve 105, wherein, for example, the sleeve 105 is color coded to indicate a property of the corresponding fiber tip 55. The sleeve 105 in the illustrated embodiment comprises a groove 110 that may be used to extract the sleeve 105 from the distal portion 50 of the electromagnetic-energy outputting handpiece 20 using a tip removing tool. A cavity 130 may be formed in the sleeve 105 adjacent to the fiber tip 55, such that the cavity 130 may accept glue that operates to secure a position of the fiber tip 55. The sleeve 105 in the illustrated embodiment further comprises a plurality of ring-shaped projections 115 that make contact with an interior of the distal portion 50 of the electromagnetic-energy outputting handpiece 20.

The sleeve 105 still further may comprise a plurality of “springing members” 120 having locking shoulders 125 that snap into recesses 52 in the distal portion 50. FIG. 3 is an on-axis view of a combination formed by the sleeve 105 and fiber tip 55 depicted in FIG. 2, further illustrating the locking shoulders 125 and gaps 131 disposed between the springing members. The diagram also illustrates a cross-section of the fiber tip 55. According to one embodiment, the sleeve 105 in FIG. 2 may be referred to as a tip ferrule.

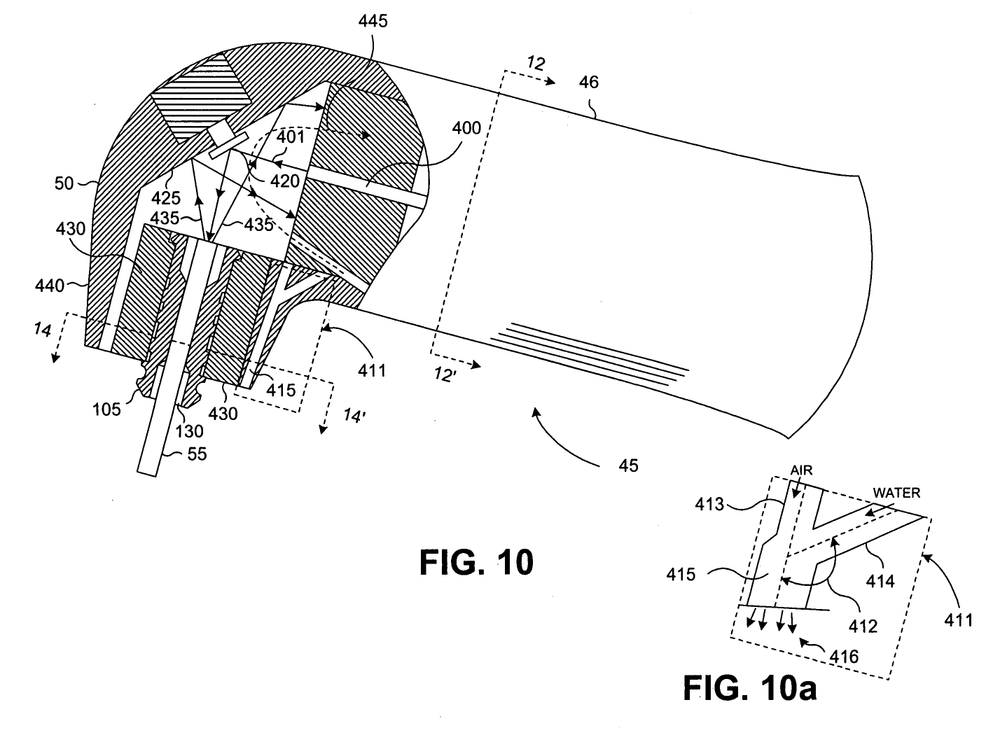

An embodiment of the distal portion 50 of the electromagnetic-energy outputting handpiece 20 comprises a plurality of mixing chambers (only one of which is illustrated in the cross-section of FIG. 2) that may receive air from the electromagnetic-energy outputting handpiece 20 through air intakes 413 and water intakes 414. As is more particularly described below with reference to FIGS. 10 and 10a, air and water may be combined in the mixing chamber and expelled through a fluid output 415, thereby generating, for example, atomized fluid particles.

FIG. 4 is a pictorial diagram of an output attachment 100 according to an example of the present invention. The diagram, which provides another view of the fiber tip 55 and sleeve 105 (i.e., tip ferrule) shown in FIG. 2 further illustrating the fiber tip 55, the groove 110, the ring-shaped projections 115, the springing members 120, and the locking shoulders shown in FIGS. 2 and 3.

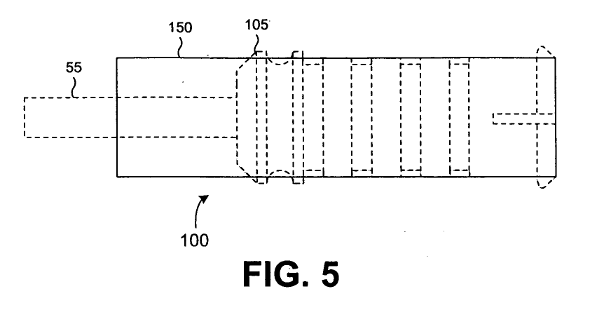

Another embodiment of an output attachment 100 may comprise, for example, a tip structure 150 as illustrated in FIG. 5. The tip structure 150 in FIG. 5 is shown with a sleeve 105 and fiber 55 superimposed in phantom. According to another embodiment, the fiber tip 55 may comprise, for example, waveguides 430 (cf. FIGS. 13 and 14), or other structures or attachments. As an example, color codes can be disposed on (e.g., integrally formed with) one or more of the sleeve 105, the fiber tip 55, and glue disposed in the cavity 130.

In a particular implementation, properties (e.g., dimensions) of housings are the same or substantially the same, while properties (e.g., dimensions) of the waveguides vary and are reflected by varying color or topography codes of the respective housings to which the waveguides are attached. For example, diameters of the housings (e.g., sleeve 105) can be the same or substantially the same, while diameters of the waveguides (e.g., fiber tip 55) can vary and be reflected by varying color codes of the respective housings to which the waveguides are attached.

Color coding of output attachments can attenuate or eliminate an error of, for example, using an unintended waveguide (e.g., fiber tip 55) type or size, or even using a waveguide in an improper application. The fiber tips may be color coded to indicate fiber size in addition to, or as an alternative to, color coding of the sleeves. In a surgical procedure, for instance, this feature can eliminate a need for a nurse or surgeon to, for example, correlate numerical dimensions with tiny objects, thereby potentially conserving operating time and costs and, further, possibly increasing convenience and reliability.

Each sleeve 105 can be formed of, for example, a plastic that is colored according to a diameter of a fiber tip held by the sleeve. Table 1 illustrates a collection of colors that may be associated with sleeves along with corresponding fiber tip diameters. As suggested by Table 1, colors are placed into one-to-one correspondence with fiber tip diameters, thereby forming a code that may describe a set of interchangeable attachments to an electromagnetic energy device (e.g., an electromagnetic-energy outputting handpiece 20 (FIG. 1)). In the illustrated example, a first sleeve may comprise a color code of purple indicating that an attached fiber tip has a diameter of 100 μm, while a second sleeve may comprise a color code of white indicating an attached fiber tip diameter of 150 μm. Continuing with the illustrated example, a third sleeve comprises a color code of yellow indicating that a 200 μm diameter fiber tip is attached thereto, a fourth sleeve comprises a color code of red corresponding to an attached fiber tip diameter of 250 μm, a fifth sleeve comprises a color code of blue indicating that an attached fiber tip has a diameter of 300 μm, a sixth sleeve comprises a color code of green according to an attached fiber tip having a diameter of 350 μm, and a seventh sleeve comprises a color code of black indicating that the attached fiber tip has a diameter of 400 μm. An eighth sleeve in the illustrated example comprises a color code of magenta corresponding to an attached fiber tip that is 600 μm in diameter.

TABLE 1

| Color | Diameter (μm) |
|---|---|
| Purple | 100 |
| White | 150 |
| Yellow | 200 |
| Red | 250 |
| Blue | 300 |
| Green | 350 |
| Black | 400 |
| Magenta | 600 |

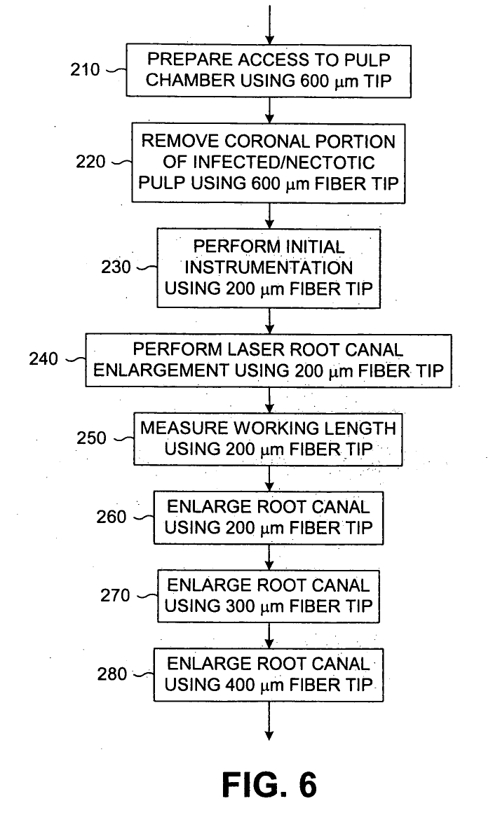

FIG. 6 is a flow diagram describing an exemplary operation that may be performed using an electromagnetic-energy outputting device more particularly described in User Manual for a Waterlase® All-Tissue Laser for Dentistry (referenced herein as “the Waterlase® User Manual” ), the entire contents of which are incorporated herein by reference. The flow diagram of FIG. 6, which is abstracted from Steps 1-8 of a root canal clinical protocol described in Appendix C of the Waterlase® User Manual, involves an implementation of a combination of different fiber tips. The fiber tips generally differ one from another in one or more of physically-discernable properties and functional properties. In accordance with an aspect of the present invention, one or more of the combinations of different fiber tips (and/or information pertaining thereto), in whole or in part, may be stored or provided as user instructions. The instructions enable a user to switch among different fiber tips during or between procedures, to thereby facilitate accomplishment of one or multiple procedures with one or more of an enhanced speed and an enhanced reliability. Efficiency and accuracy may be increased in, for example, a context of relatively complex operations that implement combinations of procedures entailing different output attachment (e.g., tip) types.

The exemplary operation may be implemented using a cutter, such as a Waterlase® laser, with certain tip types for certain procedures of the operation and other tip types for other procedures of the operation. In addition to the below exemplary operation in the context of modifying root canal surfaces, other examples can include modification of prepared cavity surfaces, modification of root surfaces at or below the gum line, and apicoectomy procedures, to name just a few.

Steps 1-8 of the root canal clinical protocol described in Appendix C of the attached reference manual may be performed by way of a user progressing through, for example, a 600 μm diameter fiber tip, a 200 μm diameter fiber tip, a 320 μm diameter fiber tip and a 400 μm diameter fiber tip. Operating parameters for steps 1-8 as delineated in the reference manual (e.g., power, pulse repetition rate, energy per pulse, water percentage and/or air percentage, and, as another option, pulse length) may be entered manually or may be recalled using presets, as disclosed in co-pending U.S. application Ser. No. 11/203,400, filed Aug. 12, 2005 and entitled DUAL PULSE-WIDTH MEDICAL LASER WITH PRESETS.

Moreover, a sequence may be programmed into the electromagnetic-energy outputting base unit 30 (FIG. 1) that progresses the user through part or substantially all steps (e.g., steps 1-8) of the procedure. In a representative embodiment, the user is prompted through the steps by way of information provided on a monitor or other visual user interface (not shown) of the electromagnetic-energy outputting base unit 30.

In accordance with an aspect of the present invention, the monitor may instruct the use of various output attachments at varying times in a given operation or procedure by visually indicating color codes or topography codes indicative of output tips which may be used at various times. For instance, each color code may be indicated by text, colored text, a color patch on the monitor, or, as another example, a background color of the monitor. The color or topography code indications may be provided in a single display or may be sequentially provided through a progression of displays. According to one example, the user is prompted through a given operation or procedure.

For example, in the context of implementing part or all of steps 1-8 of the root canal clinical protocol described in Appendix C of the Waterlase® User Manual as abstracted in steps 210-280 of FIG. 6, a first display on, for example, an electromagnetic-energy outputting base unit 30 (FIG. 1) may instruct that the user prepare access to the pulp chamber at step 210 using a 600 μm fiber tip at a setting (manual or preset) of operating parameters appropriate for cutting enamel and dentin. The first display or a subsequent display may instruct that the user then use the same fiber tip at step 220 to remove the coronal portion of the infected/necrotic pulp at an appropriate (manual or preset) setting of operating parameters. The same or a subsequent display may instruct that the user perform initial instrumentation using a 200 μm fiber tip at step 230 using an appropriate setting (manual or preset) of operating parameters. The same or subsequent displays may instruct the user then to perform laser root canal enlargement at step 240 using a 200 μm fiber tip. A similar display or displays may instruct the user to measure working length using a 200 μm fiber tip at step 250 and to enlarge the root canal at step 260 using the 200 μm fiber tip. At step 270 the user may be instructed by the same or subsequent displays to enlarge the root canal using a 300 μm fiber tip. The same or subsequent displays then may instruct the user to enlarge the root canal using a 400 μm fiber tip at step 280. All of the preceding displays further may instruct the user to use appropriate operating parameter settings (e.g., manual or preset values for power, pulse duration, pulse repetition rate, and settings for water and air). As may be appreciated, performing the steps outlined in FIG. 6 using various output attachments may require extra effort or precision on the part of the user to assure that proper output attachments are implemented at each step. Keeping in mind that a typical output attachment is quite small, extra care should be taken to assure that the correct output attachment is used at every step.

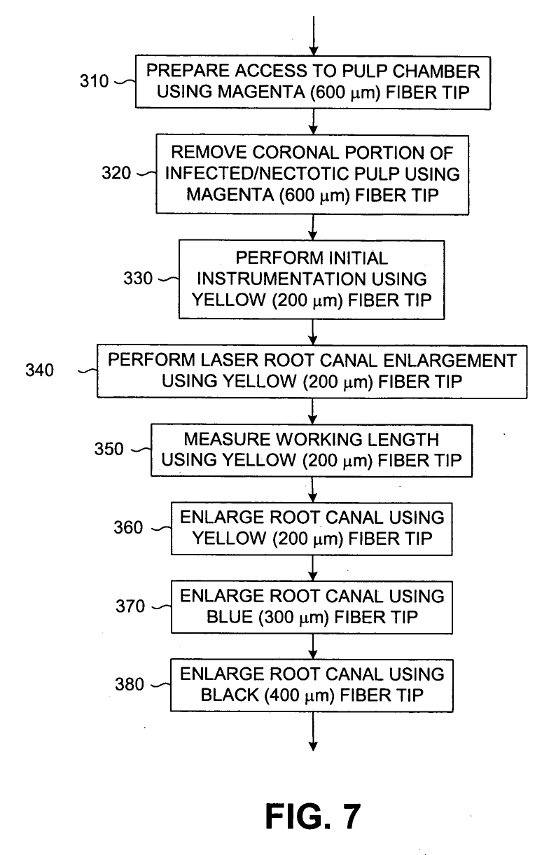

An implementation of a method of the present invention that addresses concerns with regard to, for example, small size and possible ambiguity in identifying output attachments is illustrated in the flow diagram of FIG. 7. According to the illustrated implementation, a first display instructs a user at step 310 to prepare access to the pulp chamber using a magenta (600 μm) fiber tip at a setting (manual or preset) of operating parameters appropriate for cutting enamel and dentin. The same or a subsequent display may, at step 320, instruct the user to use the magenta (600 μm) fiber tip at an appropriate setting (manual or preset) of operating parameters to remove the coronal portion of infected/necrotic pulp. Another or the same display then may instruct the user to perform initial instrumentation using a yellow (200 μm) fiber tip at step 330. Similarly, the same or subsequent displays then may instruct the user to perform laser root canal enlargement at step 340 using a yellow (200 μm) fiber tip. A subsequent or the same display may instruct the user then to measure working length using a yellow (200 μm) fiber tip at step 350. The user then may be instructed to enlarge the root canal using the yellow (200 μ) fiber tip at step 360 and then, at step 370, to enlarge the root canal using a blue (300 μm) fiber tip. The same display or a subsequent display may instruct the user to then enlarge the root canal using a black (400 μm) fiber tip. It is to be understood that the aforementioned displays may instruct the user to perform the indicated steps using appropriate (manual or preset) settings.

When more than one display is used to progress the user through part or substantially all of the steps (e.g., steps 310-380) of a given procedure, each display may progress to a subsequent display upon the detection of a user action by, for example, the electromagnetic-energy outputting base unit 30 (FIG. 1). The user action may comprise, for example, one or more of the user selecting a key or icon, or, for example, the user removing an output attachment (e.g., fiber tip) associated with a prior process or step wherein the removal is detected by the electromagnetic-energy outputting base unit 30. The detection may be accomplished using sensors (not shown) in the distal portion 50 of the electromagnetic-energy outputting handpiece 20 (FIG. 1) according to one embodiment. Moreover, the electromagnetic-energy outputting base unit 30 in conjunction with the electromagnetic-energy outputting handpiece 20 can be configured to sense a color or topography code of an output attachment (e.g., the color of a sleeve or a fiber tip) connected to the electromagnetic-energy outputting handpiece 20. The sensing may, for example, help to facilitate or ensure that the output attachment is appropriate for the settings or procedure being implemented.

In some implementations, output attachments may comprise dual codes to indicate to a user not only information pertaining to the output attachment containing the dual code(s) but also information pertaining to an electromagnetic-energy outputting handpiece or another output attachment. For example, in contexts wherein multiple output attachments are sequentially used, a proximal half or end of a first output attachment can comprise a first color, and a distal half or end of the first output attachment can comprise a second color, wherein the second color can match a color on a second output attachment that is to be subsequently used. This color progression may be propagated through additional output attachments.

In modified embodiments, any or all of the displayed information (and/or user actions) may additionally or alternatively be provided by (and/or to) the electromagnetic-energy outputting base unit in audio form via, for example, a wireless earpiece (and/or speaker).

According to one broad aspect of the present invention, at least two different color or topography codes can be used to provide visual or tactile information distinguishing among at least two different properties of waveguides held by the respective output attachments. In one implementation, at least two different color codes (e.g., solid colors or color combinations) can be used to provide visual differentiation among at least two different properties (e.g., dimensions) of the waveguides held by the respective output attachments. For instance, at least two colors (e.g., purple and white) can be used on output attachments to differentiate among at least two different dimensions (e.g., 100 μm and 150 μm) of the waveguides held by the respective output attachments. In another implementation, at least two different topography codes (e.g., surface disturbances, patterns or features) can be used to provide tactile and/or visual information distinguishing between at least two different waveguides with different properties held by the respective output attachments. As an example, at least two surfaces (e.g., smooth and textured) can be used on output attachments to differentiate among at least two different dimensions (e.g., 100 μm and 150 μm) of the waveguides held by the respective output attachments. The number of different color or topography codes (or color and topography code combinations) can correspond to or equal the number of different properties of the output attachments. This arrangement can define a one-to-one correspondence, whereby each different output attachment can be identified by a unique color or topography code (or combination of color and topography codes). In the above examples, the at least two items may further be a plurality of items according to, for example, codes illustrated in Table 1.

In certain implementations, different handpieces are utilized instead of or in addition to different output attachments. Color and/or topography codes may be placed on one or more of the different output attachments and/or different electromagnetic-energy outputting handpieces. For example, different properties of different output attachments may comprise compatibilities of the output attachments with different electromagnetic-energy outputting handpieces. As one permutation, lower-cost electromagnetic-energy outputting handpieces may accommodate a first predetermined set of output attachments, and higher-end electromagnetic-energy outputting handpieces may accommodate a second predetermined set of output attachments. The second predetermined set of output attachments may, for example, include all of the output attachments of the first predetermined set. As an example of one implementation, the codes may be implemented in the context of U.S. application Ser. No. 11/181,373, filed Jul. 13, 2005 and entitled FIBER TIP DETECTOR APPARATUS. According to the referenced application, different electromagnetic-energy outputting handpieces are programmed to accept different output attachments. The codes of the present invention may readily indicate to a user which output attachments may be used with which electromagnetic-energy outputting handpieces. Such codes may be placed on part or all of the output attachments and on part or all of the electromagnetic-energy outputting handpieces. In embodiments wherein codes are placed on some or all of the output attachments and their matching electromagnetic-energy outputting handpieces, the codes may be similar or the same between matching output attachments and electromagnetic-energy outputting handpieces to indicate compatibility. As one implementation, some output attachments may not be coded to indicate compatibility with certain non-coded electromagnetic-energy outputting handpieces. As another implementation, some output attachments may not be coded to indicate compatibility with certain non-coded and/or coded electromagnetic-energy outputting handpieces. Other output attachments may be coded to indicate compatibility with certain non-coded and/or coded electromagnetic-energy outputting handpieces. In an exemplary embodiment, codes on output attachments may have the same color as codes on electromagnetic-energy outputting handpieces to indicate compatibility. The colors of the codes and/or shapes, orientations, or positions of the codes on the output attachments and/or electromagnetic-energy outputting handpieces may indicate compatibilities between the two. For example, a single stripe of color disposed on an output attachment may indicate compatibility of that output attachment with an electromagnetic-energy outputting handpiece having a single stripe of color (e.g., of the same, similar, or different color). Also, a double stripe of color disposed on an output attachment may indicate compatibility of that output attachment with an electromagnetic-energy outputting handpiece having a double stripe of color (e.g., of the same, similar, or different color). As mentioned, in addition to numbers and/or colors of stripes, different thicknesses and/or spacings of stripes, or other shapes, may also be used to indicate compatibilities.

The various examples of the preceding paragraph regarding compatibility properties may additionally or alternatively be implemented in whole or in part in additional embodiments, such as, for example, those disclosed herein, to designate other properties of the output attachments and/or electromagnetic-energy outputting handpieces. In other implementations, color or topography codes may be used to indicate maintenance or related issues (e.g., when a pulse counter determines that an output attachment has been used a predetermined amount measured in pulse transmissions, the electromagnetic-energy outputting handpiece may apply a code, such as the color red, to the output attachment to thereby indicate a recommended replacement). In other implementations, color or topography codes may be used to indicate procedure types. For example, output attachments of a first group or type (e.g., various fiber tips 55 (FIG. 2)) may have color and/or topography codes, and output attachments of a second type (e.g., tip structures 150 (FIG. 5)) may have different (or may be devoid of) color and/or topography codes. Fiber tips for a root canal procedure, for example, may all have a common color code.

In other implementations, color or topography codes may be used to indicate operation or target-tissue types. For example, output attachments of a first group or type may have color and/or topography codes to indicate suitability for hard tissue procedures. Output attachments of a second type may have different (or may be absent of) color and/or topography codes to indicate suitability for soft tissue procedures, and/or output attachments may have color and/or topography codes to indicate suitability for hard and soft tissue procedures. In still other implementations, color or topography codes may be used to indicate operating parameter suitabilities of electromagnetic-energy outputting housings. For example, output attachments of a first group or type may have color and/or topography codes to indicate suitability for long-pulse procedures, and output attachments of a second type may have different (or may be absent of) color and/or topography codes to indicate suitability for short-pulse procedures. Other output attachments may have color and/or topography codes to indicate suitability for long and short pulse procedures. U.S. application Ser. No. 11/203,400, filed Aug. 12, 2005 and entitled DUAL PULSE-WIDTH MEDICAL LASER WITH PRESETS, discloses long-pulse and short-pulse configurations and methods that may be combined with the codes and related structures of the present invention.

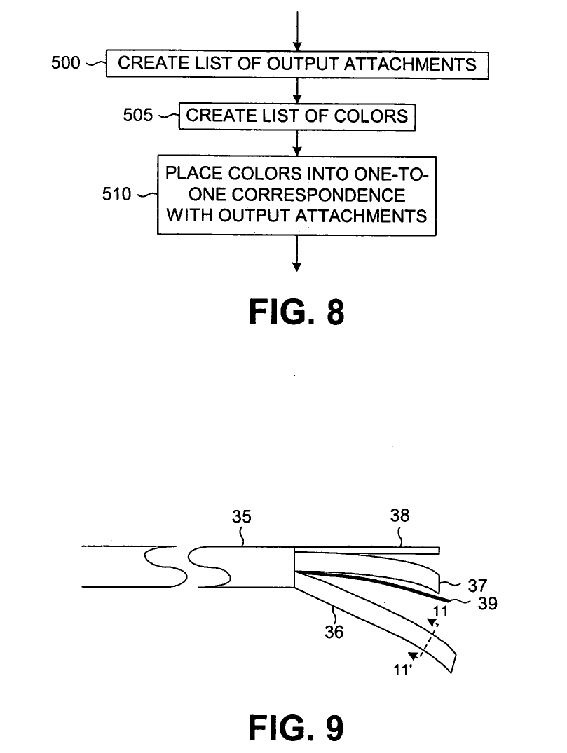

FIG. 8 is a flow diagram describing an implementation of a method of creating a code according to the present invention. The illustrated implementation comprises creating a list of output attachments at step 500. As one example, the list of output attachments may correspond to a collection of fiber tip attachments having diameters as listed in the Diameter column of Table 1. The implementation continues by creating a list of colors at step 505. An exemplary list of colors may comprise, for example, Purple, White, . . . , Magenta as illustrated in the Color column of Table 1. According to FIG. 8, the colors can then be placed into one-to-one correspondence with output attachments at step 510. Table 1 provides an example of such placing, wherein the color Purple is placed into correspondence with an output attachment having a diameter of 100 mm, the color White is placed into correspondence with an output attachment having diameter of 150 mm, and so on. In practice, each output attachment having a given diameter may be colored according to a color that corresponds to the given diameter in a table such as Table 1. The color coding may be performed by coloring, for example, a sleeve 105 of an output attachment 100 such as is shown in FIG. 4.

In an exemplary configuration, the present invention relates to an identification connector for use with U.S. application Ser. No. 11/186,619, filed Jul. 20, 2005 and entitled CONTRA-ANGLE ROTATING HANDPIECE HAVING TACTILE-FEEDBACK TIP FERRULE, the contents of which are incorporated by reference herein. An embodiment of an identification connector, described in a U.S. application Ser. No. 11/192,334, filed Jul. 27, 2005 and entitled IDENTIFICATION CONNECTOR FOR A MEDICAL LASER HANDPIECE, the entire content of which is incorporated herein by reference, may take the form shown in FIG. 1.

FIG. 9 is a pictorial diagram of another embodiment of the conduit 35 shown in FIG. 1. The illustrated embodiment of the conduit 35 includes a plurality of proximal members, such as four proximal members comprising a first proximal member 36, a second proximal member 37, a third proximal member 38, and a fourth proximal member 39. First, second, and third proximal members 36, 37, and 38 can have hollow interiors configured to accommodate one or more light transmitters or other tubular or elongate structures that have cross-sectional areas less than, for example, a cross-sectional area of a hollow interior of the conduit 35. The first, second, and third proximal members 36, 37, and 38 may be arranged such that the hollow interior of each proximal member is in communication with a hollow interior of elongate body 22 (FIG. 1). This arrangement can provide for a substantially continuous path for the light transmitters to extend from proximal portion 21 to distal portion 50 of the electromagnetic-energy outputting handpiece 20. The third proximal member 38 may receive feedback (e.g., reflected or scattered light) from the electromagnetic-energy outputting handpiece 20 and may transmit the feedback to the electromagnetic-energy outputting base unit 30 as is more particularly described below.

The fourth proximal member 39 may receive electromagnetic radiation derived from, for example, an erbium, chromium, yttrium scandium gallium garnet (Er, Cr:YSGG) solid state laser. The laser may generate electromagnetic energy having a wavelength of approximately 2.78 μm at an average power of about 6 W, a repetition rate of about 20 Hz, and a pulse width of about 150 microseconds. Moreover, electromagnetic radiation may further comprise an aiming beam, such as light having a wavelength of about 655 nm and an average power of about 1 mW transmitted in a continuous-wave (CW) mode. The fourth proximal member 39 may transmit electromagnetic radiation received from the electromagnetic-energy outputting base unit 30 to the distal portion 50 of the electromagnetic-energy outputting handpiece 20. Although the illustrated embodiment is provided with four proximal members, a greater or fewer number of proximal members may be provided in additional embodiments according to, for example, a number of light transmitters provided by the electromagnetic-energy outputting base unit 30. In addition, while the illustrated embodiment includes first and second proximal members 36 and 37 that have substantially equal diameters, and a third proximal member 38 having a diameter less than either of the diameters of the first and second proximal members 36 and 37, other diameters are also contemplated by the present invention according to modified embodiments.

FIG. 10 is a partial cut-away diagram of a handpiece tip 45 (cf. FIG. 1). The illustrated embodiment, which is enclosed by an outer surface 46, may receive electromagnetic energy, illumination light, excitation light and the like from the electromagnetic-energy outputting unit 30. Typically, the electromagnetic energy and light is transmitted through fiber waveguides, such as fibers 400 and 405 shown in FIG. 12. According to one embodiment, treatment electromagnetic energy 401 is received (e.g., through fourth proximal member 39 (FIG. 9)), carried by an internal waveguide 400, and directed toward a first mirror 420, whence reflected treatment electromagnetic energy is directed toward the fiber tip 55. Additional electromagnetic energy, such as illumination light, further may be received by the handpiece tip 45 from, for example, proximal members 36 and/or 37 (FIG. 9), carried by fibers 405 (FIG. 12), and directed toward a second mirror 425 that directs the light toward one or more waveguides 430 that may direct the light to a target area for purposes of, for example, illuminating the target area. In certain embodiments, first and second mirrors 420 and 425 may comprise parabolic, toroidal, or flat surfaces. FIG. 10 also illustrates a simplified cross-sectional view indicating a path 445 of cooling air.

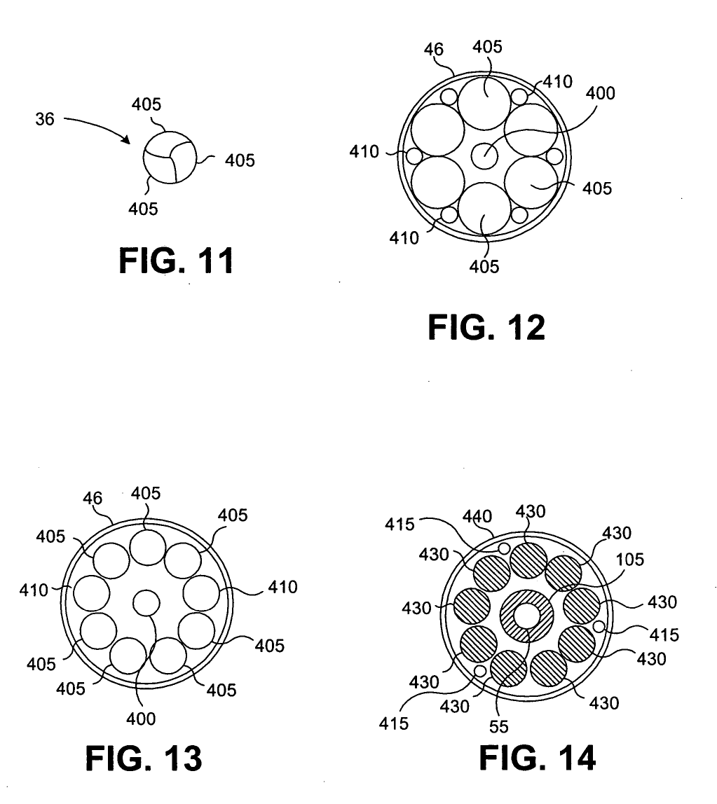

As shown in the illustrated embodiment of FIG. 11, which is a sectional view along line 11-11′ of FIG. 9, proximal member 36 (as well as proximal member 37) may comprise three optical fibers 405 substantially fused together to define a unitary light emitting assembly or waveguide. In modified embodiments, the three optical fibers 405 may be joined by other means or not joined. According to another example embodiment, proximal members 36 and/or 37 may include six optical fibers 405 that may surround an electromagnetic energy waveguide 400 as illustrated in the cross-sectional view of FIG. 12, which is taken along line 12-12′ of the handpiece tip 45 in FIG. 10. Proximal member 38 may include six relatively small fibers 410, as likewise shown in the cross-sectional view of FIG. 12. One or more additional waveguides, such as fibers corresponding to fibers 410, may be disposed within the outer surface 46, and may be configured, for example, to receive feedback light from a target surface. For example, feedback light may comprise scattered light 435 (FIG. 10) received from the fiber tip 55 in a manner more particularly described below. The scattered light 435 (e.g., feedback light) may be transmitted by, for example, the third proximal member 38 (FIG. 9) to the electromagnetic-energy outputting base unit 30 (FIG. 1). Fibers 410 are illustrated in FIG. 12 as being separate from each other, but in additional embodiments, two or more of the fibers 410 may be fused or otherwise joined together. Fibers 405 and 410 can be manufactured from plastic using conventional techniques, such as extrusion and the like.

FIG. 13 is a cross-sectional diagram of another embodiment of the handpiece tip 45 taken along the line 12-12′ of FIG. 10. FIG. 13 depicts a treatment electromagnetic energy waveguide 400 surrounded by illumination waveguides (e.g., fibers 405) and feedback waveguides (e.g., fibers 410) all of which are disposed within outer surface 46. In a manner similar to that described above with reference to FIG. 12, the illumination waveguides (e.g. fibers 405) may receive light energy from an electromagnetic-energy outputting base unit by way of proximal members 36 and/or 37 (FIG. 9) and may direct the light to the distal portion 50 of the electromagnetic-energy outputting handpiece 20. The feedback waveguides (e.g., fibers 410) may receive light energy from the fiber tip 55 (FIG. 10) and may transmit the light to the electromagnetic-energy outputting base unit 30 (FIG. 1). In other embodiments, described, for example, in the above-referenced U.S. application Ser. No. 11/192,334, filed Jul. 27, 2005 and entitled IDENTIFICATION CONNECTOR FOR A MEDICAL LASER HANDPIECE, the electromagnetic-energy outputting base unit 30 may additionally supply spray air, spray water, and/or cooling air to the electromagnetic-energy outputting handpiece 20.

FIG. 14 is a cross-sectional diagram of another embodiment of the handpiece tip 45 taken along line 14-14′ of FIG. 10. The embodiment illustrates a fiber tip 55 surrounded by a sleeve 105, and, optionally, glue that fills a cavity 130 around the fiber tip 55 to hold the fiber tip 55 in place. Waveguides 430 may receive illumination from the illumination fibers 405 and direct the illumination to a target. In certain embodiments, fluid outputs 415 are disposed in the handpiece tip 45. The fluid outputs 415 may carry, for example, air and/or water. Illumination exiting from the illumination fibers 405 (cf. FIG. 13) can be reflected by mirror 425 (cf. FIG. 13) into the waveguides 430 (cf. FIGS. 13 and 14). While a portion of this illumination may also be reflected by mirror 425 into fiber tip 55, fiber tip 55 receives, primarily, relatively high energy 401 from treatment electromagnetic-energy emitting fiber 400 (cf. FIG. 13), which, as presently embodied, comprises both treatment beam and aiming beam electromagnetic radiation. In a representative embodiment, light from the illumination fibers 405 that exits the waveguides 430 is white light of variable intensity for facilitating viewing and examination of individual places of a target surface, such as a tooth, more closely. For example, a cavity in a tooth may be closely examined and treated under the aid of light from the multiple waveguides 430.

A detailed illustration of an embodiment of a chamber for mixing spray air and spray water in the handpiece tip 45 is illustrated in FIG. 10a. As shown, the mixing chamber comprises an air intake 413 connected to, for example, tubing (not shown) that connects to and receives air from a spray air connection in the connector 40 (FIG. 1). Similarly, a water intake 414 may connect to tubing (not shown) that connects to and receives water from a spray water connection in the connector 40 (FIG. 1). The air intake 413 and the water intake 414, which may have circular cross-sections about 250 μm in diameter, join at an angle 412 that may approximate, for example, 110° in a typical embodiment. Mixing may occur in a neighborhood where the air intake 413 and water intake 414 join, and a spray (e.g., atomized) mixture 416 of water and air may be ejected through a fluid output 415. The embodiment illustrated in FIG. 14 depicts three fluid outputs 415. These fluid outputs may, for example, correspond to, comprise parts of, or comprise substantially all of any of the fluid outputs described in U.S. application Ser. No. 11/042,824, filed Jan. 24, 2005 and entitled ELECTROMAGNETICALLY INDUCED TREATMENT DEVICES AND METHODS, the entire contents of which are incorporated herein by reference, to the extent compatible, or, in other embodiments, structures described in the referenced provisional patent application may be modified to be compatible with the present invention. The fluid outputs 415 may, as illustrated in FIGS. 10, 10a and 14, have circular cross-sections measuring about 350 μm in diameter.

Scattering of the aiming beam can be detected and analyzed to monitor, for example, integrity of optical components that transmit the treatment and aiming beams. For example, in typical implementations the aiming beam has little to no reflection back into the feedback fibers 410, but if any components (such as, for example, mirror 420 or fiber tip 55) are damaged, scattering of the aiming beam light (which may be red in some embodiments) may occur. Scattered light 435 (FIG. 10) may be directed into feedback fibers 410 that may carry the scattered light to the electromagnetic-energy outputting base unit 30 (FIG. 1) where it can be detected by a photo detector that forms an electrical signal corresponding to radiation supplied to it. Detection of scattered aiming beam light beyond a predetermined threshold can trigger the electromagnetic-energy outputting base unit 30 or other structure to provide an error or potential error indication. The magnitude of detected scattered light 435 from the feedback fibers 410 and/or relative magnitudes of detected scattered light among or between the various feedback fibers 410 can be automatically analyzed and/or compared with predetermined optical-component failure criteria to provide additional information to a user regarding a type and severity of the potential optical-component problem. All of these calculations can be based on the color (type) of the fiber tip 55. Feedback can be provided on a monitor of the electromagnetic-energy outputting base unit (e.g., a color of blue) to indicate one or more of the above-stated indications or parameters.

In one embodiment, the waveguides 430 and sleeve 105 are housed (e.g., supported) in a housing 440 which may comprise, for example, metal. According to one implementation, an interior of the housing 440 is solid, with cavities disposed therein for accommodating, for example, the sleeve 105 and/or waveguides 430 and for defining the fluid outputs 415. In other implementations, the housing 440 and/or interior comprise a transparent material, such as a transparent plastic, sapphire, or quartz, so that the waveguides 430 may optionally be omitted. Thus, in some embodiments, light can be transmitted through the transparent material of the interior without a need for disposing or defining waveguides 430, so that the interior may comprise cavities only for the sleeve 105 and fluid outputs 415.

The embodiment of FIG. 13 shows an electromagnetic-energy emitting fiber 400 surrounded by six illumination (and/or “excitation” in certain implementations involving, for example, caries detection, as disclosed in related U.S. application Ser. No. 11/203,399, filed Aug. 12, 2005) fibers 405 and three feedback fibers 410. In other embodiments, greater or fewer numbers, or different dimensions or spacings, of the illumination fibers 405 (and/or waveguides 430) and/or feedback fibers 410 may be used. According to one aspect of the invention, two or more of each, and preferably three or more of each, may be used, for example, to avoid shadowing, which may be caused by using, for example, only a single (or, in some implementations, two) illumination fiber(s) 405 and/or waveguide(s) 430.

In a representative embodiment, the fluid outputs are positioned at about zero, one hundred twenty, and two hundred forty degrees, i.e., at a spacing of about one hundred twenty degrees. In another embodiment, the six illumination fibers 405 and three feedback fibers 410 (FIG. 13) are optically aligned with and coupled, for example, on a one-to-one basis, via mirror 425, to the nine waveguides 430 (FIG. 14). For example, if the nine elements (i.e., the six illumination fibers 405 and three feedback fibers 410 in FIG. 12) are approximately evenly spaced and disposed at about zero, forty, eighty, one hundred twenty, one hundred sixty, two hundred, two hundred forty, two hundred eighty, and three hundred twenty degrees, then the nine waveguides 430 also may be approximately evenly spaced and disposed at about zero, forty, eighty, one hundred twenty, one hundred sixty, two hundred, two hundred forty, two hundred eighty, and three hundred twenty degrees. In another embodiment wherein, for example, the waveguides 430 are arranged in relatively closely-spaced groups of three with each group being disposed between two fluid outputs, the waveguides 430 may be disposed at, for example, about zero, thirty-five, seventy, one hundred twenty, one hundred fifty-five, one hundred ninety, two hundred forty, two hundred seventy-five, and three hundred ten degrees. In one such embodiment, the waveguides 430 may likewise be disposed at about zero, thirty-five, seventy, one hundred twenty, one hundred fifty-five, one hundred ninety, two hundred forty, two hundred seventy-five, and three hundred ten degrees. Further, in such an embodiment, the fluid outputs may be disposed between the groups of waveguides at about ninety-five, two hundred fifteen, and three hundred thirty-five degrees.

The cross-sectional views of FIGS. 12 and 13 may alternatively (or additionally), without being changed, correspond to line 12-12′ taken in FIG. 10 closer to (or next to) the mirrors 420 and 425 to elucidate corresponding structure that outputs radiation distally onto the mirrors 420 and 425. The diameters of the illumination fibers 405 and feedback fibers 410 may be different (cf. the cross-sectional view of FIG. 12) or the same or substantially the same (cf. the cross-sectional view of FIG. 13). In-an exemplary embodiment, the illumination fibers 405 and feedback fibers 410 in FIG. 13 comprise plastic constructions with diameters of about 1 mm, and the waveguides 430 in FIG. 14 comprise sapphire constructions with diameters of about 0.9 mm.

Corresponding or related structure and methods described in the following patents assigned to BioLase Technology, Inc., are incorporated herein by reference in their entireties, wherein such incorporation includes corresponding or related structure (and modifications thereof) in the following patents which may be (i) operable with, (ii) modified by one skilled in the art to be operable with, and/or (iii) implemented/used with or in combination with any part(s) of, the present invention according to this disclosure, that/those of the patents, and the knowledge and judgment of one skilled in the art: U.S. Pat. No. 5,741,247; U.S. Pat. No. 5,785,521; U.S. Pat. No. 5,968,037; U.S. Patent No. 6,086,367; U.S. Pat. No. 6,231,567; U.S. Pat. No. 6,254,597; U.S. Pat. No. 6,288,499; U.S. Pat. No. 6,350,123; U.S. Pat. No. 6,389,193; U.S. Pat. No. 6,544,256; U.S. Pat. No. 6,561,803; U.S. Pat. No. 6,567,582; U.S. Pat. No. 6,610,053; U.S. Pat. No. 6,616,447; U.S. Pat. No. 6,616,451; U.S. Pat. No. 6,669,685; U.S. Pat. No. 6,744,790 and U.S. Pat. No. 6,821,272. For example, output optical energy distributions may be useful for optimizing or maximizing a treatment or cutting effect of an electromagnetic energy source, such as a laser. The electromagnetic energy output can be directed, for example, into fluid (e.g., an atomized distribution of fluid particles) above a target surface. An apparatus for directing electromagnetic energy into an atomized distribution of fluid particles above a target surface is disclosed in the above-referenced U.S. Pat. No. 5,574,247. The laser can impart large amounts of energy into the fluid (e.g., atomized fluid particles) which can comprise water, to thereby expand the fluid (e.g., fluid particles) and apply disruptive (e.g., mechanical) cutting forces to the target surface.

It will be appreciated and understood that the method and apparatus described herein are intended to be operable with devices and methods described in the Waterlase® User Manual, U.S. application Ser. No. 11/181,373, filed Jul. 13, 2005 and entitled FIBER TIP DETECTOR APPARATUS, U.S. application Ser. No. 11/186,619, filed Jul. 20, 2005 and entitled CONTRA-ANGLE ROTATING HANDPIECE HAVING TACTILE-FEEDBACK TIP FERRULE, U.S. application Ser. No. 11/191,594, filed Jul. 27, 2005 and entitled DUAL PULSE-WIDTH MEDICAL LASER, U.S. application Ser. No. 11/192,329, filed Jul. 27, 2005 and entitled MEDICAL LASER HAVING CONTROLLED-TEMPERATURE AND STERILIZED FLUID OUTPUT, U.S. application Ser. No. 11/192,334, filed Jul. 27, 2005 and entitled IDENTIFICATION CONNECTOR FOR A MEDICAL LASER HANDPIECE, U.S. application Ser. No. 11/203,677, filed Aug. 12, 2005 and entitled LASER HANDPIECE ARCHITECTURE AND METHODS, U.S. application Ser. No. 11/203,399, filed Aug. 12, 2005 and entitled CARIES DETECTION USING TIMING DIFFERENTAILS BETWEEN EXCITATION AND RETURN PULSES, and U.S. application Ser. No. 11/203,400, filed Aug. 12, 2005 and entitled DUAL PULSE-WIDTH MEDICAL LASER WITH PRESETS, all of which are commonly assigned and the entire contents of which are incorporated herein by reference.

In view of the foregoing, it will be understood by those skilled in the art that the methods and apparatus of the present invention can facilitate operation of electromagnetic-energy outputting devices, in particular electromagnetic-energy outputting devices to which may be attached a plurality of different output attachments. While this invention has been described with respect to various specific examples and embodiments, it is to be understood that the invention is not limited thereto and that it can be variously practiced. Multiple variations, combinations and modifications to the disclosed embodiments will occur, to the extent not mutually exclusive, to those skilled in the art upon consideration of the foregoing description. Additionally, other combinations, omissions, substitutions and modifications will be apparent to the skilled artisan in view of the disclosure herein. Accordingly, the present invention is not intended to be limited by the disclosed embodiments, but is to be defined by reference to the appended claims.

What is claimed is:

1. An electromagnetic-energy outputting device, comprising:
an electromagnetic-energy outputting base unit configured to provide a supply electromagnetic-energy;
an electromagnetic-energy outputting handpiece constructed to be coupled to the electromagnetic-energy outputting base unit and to receive electromagnetic-energy from the electromagnetic-energy outputting base unit; and
a plurality of output attachments that are structured to be interchangeably coupled to the electromagnetic-energy outputting handpiece, wherein a coupled output attachment is adapted to receive electromagnetic-energy from the electromagnetic-energy outputting handpiece and to deliver electromagnetic-energy to a target site, members of the plurality of output attachments being distinguishable one from another according to one or more of a physically-discernable property and a functional property being coded according to one or more of the physically-discernable property and the functional property.

2. The electromagnetic-energy outputting device as set forth in claim 1, wherein:
members of the plurality of output attachments are coded according to a physically-discernable property; and
the physically-discernable property comprises a diameter of an output attachment.

3. The electromagnetic-energy outputting device as set forth in claim 2, wherein the members of the plurality of output attachments comprise waveguides.

4. The electromagnetic-energy outputting device as set forth in claim 2, wherein:
members of the plurality of output attachments are coded according to a functional property; and
the functional property comprises a rating of an output attachment.

5. The electromagnetic-energy outputting device as set forth in claim 4, wherein the members of the plurality of output attachments comprise waveguides.

6. The electromagnetic-energy outputting device as set forth in claim 5, wherein the rating comprises a recommended number of electromagnetic-energy pulses received by a waveguide of the output attachment before replacement.

7. The electromagnetic-energy outputting device as set forth in claim 4, wherein the electromagnetic-energy outputting device comprises one or more of the following:
a list of physically-discernable properties placed in one-to-one correspondence with one or more of a list of colors and a list of topographies, thereby defining one or more of a physically-discernable color code and a physically-discernable topography code; and
a list of functional properties placed in one-to-one correspondence with one or more of a list of colors and a list of topographies, thereby defining one or more of a functional color code and a functional topography code.

8. The electromagnetic-energy outputting device as set forth in claim 7, wherein:
the physically-discernable color code comprises a list of waveguide diameters placed in one-to-one correspondence with a list of colors;
the physically-discernable topography code comprises a list of waveguide diameters placed in one-to-one correspondence with a list of topographies;
the functional color code comprises a list of ratings placed in one-to-one correspondence with a list of colors; and
the functional topography code comprises a list of ratings placed in one-to-one correspondence with a list of topographies.

9. The electromagnetic-energy outputting device as set forth in claim 8, wherein:
the list of waveguide diameters comprises one or more of a 100 μm value, a 150 μm value, a 200 μm value, a 250 μm value, a 300 μm value, a 350 μm value, a 400 μm value, and a 600 μm value; and
the list of colors comprises one or more of a purple color, a white color, a yellow color, a red color, a blue color, a green color, a black, and a magenta color.

10. A code describing interchangeable output attachments to an electromagnetic-energy outputting device, the code comprising:
a list of output attachments distinguishable according to one or more of a physically-discernable property and a functional property; and
a list of color codes, wherein the color codes are placed into one-to-one correspondence with the output attachments according to one or more of the physically-discernable property and the functional property.

11. The code as set forth in claim 10, wherein the physically-discernable property comprises a waveguide diameter.

12. The code as set forth in claim 10, wherein the functional property comprises a rating according to a recommended number of electromagnetic-energy pulses received by a waveguide of an output attachment before replacement.

13. The code as set forth in claim 10, further comprising a list of topographies, wherein the topographies are placed into one-to-one correspondence with the output attachments according to a functional property.

14. The code as set forth in claim 10, further comprising a list of topographies, wherein the topographies are placed into one-to-one correspondence with the output attachments according to a physically-discernable property.

15. The code as set forth in claim 14, wherein the physically-discernable property comprises a waveguide diameter.

16. A method of coding interchangeable output attachments that attach to an electromagnetic-energy outputting device, the method comprising:
creating a list of output attachments, wherein one output attachment in the list is distinguishable from another output attachment in the list according to one or more of a physically-discernable property and a functional property;
creating a list of items, which specify one or more of color codes and topography codes; and
placing the items into one-to-one correspondence with the output attachments.

17. The method as set forth in claim 16, wherein the creating of a list of output attachments comprises creating a list of output attachments distinguishable one from another according to waveguide diameters of the output attachments.

18. The method as set forth in claim 17, wherein the placing of items into one-to-one correspondence with the output attachments comprises placing an output attachment having a waveguide diameter chosen from a list of waveguide diameter values comprising 100 μm, 150 μm, 200 μm, 250 μm, 300 μm, 350 μm, 400 μm, and 600 μm into one-to-one correspondence with a color code chosen from a list of colors comprising purple, white, yellow, red, blue, green, black, and magenta.

19. The method as set forth in claim 17, wherein the placing of items into one-to-one correspondence with the output attachments comprises placing output attachments having different waveguide diameters into one-to-one correspondence with different color codes.

20. The method as set forth in claim 16, wherein the placing of items into one-to-one correspondence with the output attachments comprises placing an output attachment having a waveguide rating into one-to-one correspondence with a waveguide diameter.

21. The method as set forth in claim 16, wherein the placing of items into one-to-one correspondence with the output attachments comprises placing a plurality of output attachments having different waveguide ratings into one-to-one correspondence with a plurality of different waveguide diameters.

22. The method as set forth in claim 16, wherein the creating of a list of items is performed by creating a list of topographies and the placing is performed by placing the topographies into one-to-one correspondence with the output attachments.

23. The method as set forth in claim 16, wherein the creating of a list of items is performed by creating a list of color codes and the placing is performed by placing the color codes into one-to-one correspondence with the output attachments.

24. The method as set forth in claim 16, wherein:
the creating or a list of output attachments is performed by creating a list of output attachments wherein one output attachment in the list is distinguishable from another output attachment in the list according to a functional property;
the creating a list of items is performed by creating a list of color codes; and
the placing is performed by placing the color codes into one-to-one correspondence with the output attachments.

25. The method as set forth in claim 16, further comprising:
the creating or a list of output attachments is performed by creating a list of output attachments wherein one output attachment in the list is distinguishable from another output attachment in the list according to a functional property;
the creating a list of items is performed by creating a list of topographies; and
the placing is performed by placing the topographies into one-to-one correspondence with the output attachments.

26. A method of operating an electromagnetic-energy outputting device, comprising:
receiving at least one coded instruction by way of one or more of a display and a speaker of the electromagnetic-energy outputting device; and
connecting an output attachment to the electromagnetic-energy outputting device according to the coded instruction.

27. The method as set forth in claim 26, wherein the receiving of a coded instruction comprises receiving an instruction according to one or more of a color code of an output attachment and a topography code of the output attachment.

28. The method as set forth in claim 26, wherein the receiving of at least one coded instruction comprises receiving a sequence of coded instructions.

29. The method as set forth in claim 26, wherein the receiving of at least one coded instruction comprises receiving an instruction according to an output attachment that is coded with a first color code and a second color code.

30. The method as set forth in claim 26, wherein the receiving of at least one coded instruction comprises:
receiving a first instruction according to an output attachment having at least a first color code and a second color code; and
receiving a second instruction according to an output attachment having at least the second color code and a third color code.

31. The method as set forth in claim 26, wherein the receiving of at least one coded instruction comprises receiving a coded instruction according to an output attachment having a color code and a topography code.

32. The method as set forth in claim 26, wherein the receiving of at least one coded instruction comprises receiving a coded instruction according to at least two color codes of an output attachment, wherein the at least two color codes form a pattern.

33. The method as set forth in claim 32, wherein the receiving of a coded instruction according to at least two color codes of an output attachment comprises receiving a coded instruction wherein the at least two color codes form a striped pattern.

34. A code describing interchangeable output attachments to an electromagnetic-energy outputting device, the code comprising:
a list of output attachments distinguishable according to one or more of a physically-discernable property and a functional property; and
a list of topographies, wherein the topographies are placed into one-to-one correspondence with the output attachments according to one or more of the physically-discernable property and the functional property.

35. The code as set forth in claim 34, wherein the physically-discernable property comprises a waveguide diameter.
